(19) United States
(12) Patent Application Publication
(10) Pub. No.: US 2017/0007594 A1
(43) Pub. Date: Jan. 12, 2017

(54) THERAPEUTIC COMPOSITION AND CONFIGURATION

(71) Applicant: NICONOVUM USA, INC., Winston -Salem, NC (US)

(72) Inventor: August J. Borschke, Winston-Salem, NC (US)

(73) Assignee: NICONOVUM USA,INC

(21) Appl. No.: 14/794,026

(22) Filed: Jul. 8, 2015

(51) Int. Cl. A61K 31/465; A61K 9/00; A24F 47/00

(52) U.S. Cl. CPC A61K 31/465; A24F 47/00; A61K 9/0056

(57) ABSTRACT

A system intended to be employed for therapeutic purposes incorporates an active ingredient (e.g., a source of nicotine). Representative forms of nicotine include free base (e.g., as a mixture of nicotine and microcrystalline cellulose), a nicotine salt (e.g., as nicotine bitartrate) and nicotine polacrilex. The system preferably comprises a lozenge incorporating the active ingredient, adapted to provide oral administration of nicotine. The lozenge is in contact with a substrate (e.g., hollow tube) that can be manipulated within the mouth of the user (e.g., the hollow tube can be drawn upon to simulate the inhalation of cigarette smoke). As such, the active ingredient is administered and the user is able to experience certain other physiological sensations. The composition is useful for treatment of central nervous system conditions, diseases, and disorders, and can be used as a nicotine replacement therapy.

### FIELD OF THE INVENTION

The present invention relates to systems that contain active ingredients that can be characterized as those having a pharmacological effect and that can be considered to be useful for therapeutic purposes, and in particular, to such systems that are adapted to possess component parts or pieces having predetermined structures or shapes.

### BACKGROUND OF THE INVENTION

Central nervous system (CNS) conditions, diseases, or disorders can be drug induced; can be attributed to genetic predisposition, infection or trauma; or can be of unknown etiology. They comprise neuropsychiatric disorders, neurological diseases and mental illnesses; and include neurodegenerative diseases, behavioral disorders, cognitive disorders and cognitive affective disorders. The clinical manifestations of several CNS conditions, diseases or disorders have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors).

Nicotinic compounds, such as nicotine, are capable of affecting nicotinic acetylcholinergic receptors (nAChRs). Subtypes of nAChRs exist in both the CNS and the peripheral nervous system (PNS), but the distribution of subtypes is heterogeneous. For instance, certain subtypes are predominant in vertebrate brain, others predominate at the autonomic ganglia, and others predominate at the neuromuscular junction. Activation of nAChRs by nicotinic compounds results in neurotransmitter release. See, for example, Dwoskin et al., Exp. Opin. Ther. Patents, 10: 1561-1581 (2000); Schmitt et al., Annual Reports in Med. Chem., 35: 41-51 (2000); Huang et al., J. Am. Chem. Soc., 127: 14401-14414 (2006); Arneric et al., Biochem. Pharmacol., 74: 1092-1101 (2007) and Millar, Biochem. Pharmacol., 78: 766-776 (2009), which are incorporated herein by reference.

It has been suggested that administration of nicotine, and other nicotinic compounds, can result in various pharmacological effects. See, for example, U.S. Pat. No. 5,583,140 to Bencherif et al.; U.S. Pat. No. 5,723,477 to McDonald et al.; U.S. Pat. No. 7,001,900 to Jacobsen et al.; U.S. Pat. No. 7,135,484 to Dart et al. and U.S. Pat. No. 7,214,686 to Bencherif et al.; and US Pat. Pub. Nos. 2010/0004451 to Ahmad et al. and 2011/0274628 to Borschke; which are incorporated herein by reference. As a result, it has been suggested that nicotine, and other nicotinic compounds, can exhibit utility as active ingredients in the treatment of a wide variety of conditions, diseases, and disorders, including those that affect the CNS. Additionally, administration of nicotine and nicotinic compounds has been proposed for treatment of certain other conditions, diseases, and disorders. See, for example, U.S. Pat. No. 5,604,231 to Smith et al.; U.S. Pat. No. 5,811,442 to Bencherif et al.; U.S. Pat. No. 6,238,689 to Rhodes et al. and U.S. Pat. No. 6,489,349 to Bencherif et al., which are incorporated herein by reference. Furthermore, administration of nicotine has been employed in an effort to help cigarette smokers quit smoking (i.e., as a smoking cessation aid). For example, nicotine has been an active ingredient of various types of so-called “nicotine replacement therapy” or “NRT” products. See, for example, the background art set forth in US Pat. Pub. No. 2011/0268809 Brinkley et al., which is incorporated herein by reference.

It has been proposed to administer nicotine using a transdermal patch. Representative types of nicotine-containing transdermal patch products have been marketed under the tradenames “Habitrol,” “Nicoderm,” “Nicorette,” “Nicorette CQ,” “Nicotinell” and “ProStep.” See also, for example, U.S. Pat. No. 4,597,961 to Etscom; U.S. Pat. No. 5,298,257 to Bannon et al.; U.S. Pat. No. 5,603,947 to Wong et al.; U.S. Pat. No. 5,834,011 to Rose et al.; U.S. Pat. No. 6,165,497 to Osborne et al. and U.S. Pat. No. 6,676,959 to Anderson et al., which are incorporated herein by reference. It also has been suggested that transdermal administration of nicotine can be accompanied by ingestion of other types of nicotine-containing products. See, for example, U.S. Pat. No. 5,593,684 to Baker et al.; US Pat. Pub. No. 2009/0004249 to Gonda and Fagerstrom, Health Values, 18:15 (1994), which are incorporated herein by reference.

One particularly popular way to provide for oral administration of nicotine has been through the use of nicotine-containing gum or other type of similarly chewable product. Gum forms of product generally include a gum base (e.g., typically the types of pharmaceutically acceptable gum bases available from sources such as Gum Base Co. S.p.a., Wm. J. Wrigley Jr. Company or Gumlink A/S). See, for example, the types of nicotine-containing gums, gum formulations, gum formats and configurations, gum characteristics and techniques for formulating or manufacturing gums set forth in U.S. Pat. No. 3,845,217 to Ferno et al.; U.S. Pat. No. 3,877,468 to Lichtneckert et al.; U.S. Pat. No. 3,901,248 to Lichtneckert et al.; U.S. Pat. No. 4,317,837 to Kehoe et al.; U.S. Pat. No. 4,802,498 to Ogren; U.S. Pat. No. 5,154,927 to Song et al.; U.S. Pat. No. 6,322,806 to Ream et al.; U.S. Pat. No. 6,344,222 to Cherukuri et al.; U.S. Pat. No. 6,355,265 to Ream et al.; U.S. Pat. No. 6,358,060 to Pinney et al.; U.S. Pat. No. 6,773,716 to Ream et al.; U.S. Pat. No. 6,893,654 to Pinney et al.; U.S. Pat. No. 7,101,579 Athanikar et al.; U.S. Pat. No. 7,163,705 to Johnson et al. and U.S. Pat. No. 7,208,186 to Norman et al.; US Pat. Pub. Nos. 2004/0191322 to Hansson; 2004/0194793 to Lindell et al.; 2006/0099300 to Andersen et al.; 2006/0121156 to Andersen et al.; 2006/0165842 to Andersen et al.; 2006/0204451 to Salini; 2006/0246174 to Andersen et al.; 2006/0275344 to Mody et al.; 2007/0014887 to Cherukuri et al.; 2007/0269386 to Steen et al.; 2009/0092573 to Andersen and 2010/0061940 to Axelsson et al.; which are incorporated herein by reference. Representative nicotine-containing gum products have been marketed under the tradenames “Nicorette,” “Nicotinell” and “Zonnic.”

Another way that has been employed to provide oral administration of nicotine has been through the use of nicotine-containing lozenge or tablet types of products. Nicotine-containing lozenge, mini lozenge, tablet, and microtab types of products have been marketed under the tradenames “Commit,” “Nicorette,” “Nicotinell” and “NiQuitin.” See also, for example, U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al. and U.S. Pat. No. 6,248,760 to Wilhelmsen; US Pat. Pub. Nos. 2001/0016593 to Wilhelmsen and 2010/0004294 to Axelsson et al., which are incorporated herein by reference.

A further method that has been employed to provide oral administration of nicotine has been through the use of nicotine-containing pouches or sachet types of products. See, for example, the types of pouch materials and nicotine-containing formulations set forth in U.S. Pat. No. 4,907,605 to Ray et al. and US Pat. Pub. No. 2009/0293895 to Axelsson et al., which are incorporated herein by reference. See also, for example, the types of pouch materials and pouch manufacturing techniques (e.g., pouch filling and sealing techniques) set forth in US Pat. Pub. No. 2010/0018539 to Brinkley et al., which is incorporated herein by reference. Representative nicotine-containing pouch-type products have been marketed under the tradename “Zonnic.”

Attempts have been made to incorporate nicotine into beverages (e.g., water, juices, coffee and so-called fortified beverages). See, for example, U.S. Pat. No. 6,211,194 to Westman et al.; U.S. Pat. No. 6,268,386 to Thompon; U.S. Pat. No. 6,749,882 to Fortune, Jr.; U.S. Pat. No. 7,115,297 to Stillman and U.S. Pat. No. 7,435,749 to Knight, which are incorporated herein by reference. Additionally, attempts have been made to market nicotine-containing beverages, such as certain types of beverages have been introduced commercially under the tradenames “Nic Lite,” “Nico Water,” “Nic Med,” and Nico Shot.”

Nicotine also has been administered in inhalable form, such as in the form of nasal or oral sprays. Typically, sprays are applied within the nose or mouth for absorption through nasal or oral mucosa. Various exemplary ways to administer nicotine in the form of a nasal spray are set forth in U.S. Pat. No. 4,579,858 to Ferno et al.; U.S. Pat. No. 5,656,255 to Jones and U.S. Pat. No. 6,596,740 to Jones, which are incorporated herein by reference. Various exemplary ways to administer nicotine in the form of an oral spray, such as for buccal administration, are set forth in U.S. Pat. No. 6,024,097 to Von Wielligh; US Pat. Pub. Nos. 2003/0159702 to Lindell et al.; 2007/0163610 to Lindell et al. and 2009/0023819 to Axelsson; EP 1458388 to Lindell et al. and PCT WO 2008/037470 to Axelsson et al., which are incorporated herein by reference. Various other types of inhalable formulations, and various vapor delivery devices and systems, are set forth in U.S. Pat. No. 4,284,809 to Ray; U.S. Pat. No. 4,800,903 to Ray et al.; U.S. Pat. No. 5,167,242 to Turner et al.; U.S. Pat. No. 6,098,632 to Turner et al.; U.S. Pat. No. 6,234,169 to Bulbrook et al. and U.S. Pat. No. 6,874,507 to Fan; US Pat. Pub. Nos. 2004/0034068 to Warchol et al; 2006/0018840 to Lechuga-Ballesteros; 2008/0302375 to Andersson et al. and 2009/0005423 to Gonda, which are incorporated herein by reference. Representative nicotine-containing spray-type and inhalation types of products have been marketed under the tradenames “Favor,” “Nicotrol NS,” “Quit” and “Zonnic.”

There also have been proposed numerous smoking products, flavor generators, and medicinal inhalers that utilize electrical energy to vaporize or heat volatile materials (e.g., formulations that incorporate components such as tobacco-derived nicotine, glycerin, propylene glycol, organic acids and flavors). See, for example, the various alternative smoking articles, aerosol delivery devices and heat generating sources set forth in the background art described in U.S. Pat. No. 7,726,320 to Robinson et al.; and U.S. Pat. App. Pub. Nos. 2013/0255702 to Griffith, Jr. et al.; and 2014/0096781 to Sears et al., which are incorporated herein by reference. See also, for example, the various types of smoking articles, aerosol delivery devices and electrically powered heat generating sources referenced by brand name and commercial source in U.S. patent application Ser. No. 14/170,838, filed Feb. 3, 2014, to Bless et al. and Ser. No. 14/194,233, filed Feb. 28, 2014, to DePiano et al., which are incorporated herein by reference.

Various other ways to provide a source of nicotine, or to administer nicotine, have been proposed. For example, it has been suggested that nicotine can be incorporated into orally dissolving films (e.g., U.S. Pat. No. 6,709,671 to Zerbe et al.; U.S. Pat. No. 7,025,983 to Leung et al. and U.S. Pat. No. 7,491,406 to Leung et al.; and US Pat. Pub. Nos. 2006/0198873 to Chan et al.; 2006/0204559 to Bess et al. and 2010/0256197 to Lockwood et al.); oral osmotic devices (e.g., U.S. Pat. No. 5,147,654 to Place et al.); gum pads (e.g., U.S. Pat. No. 6,319,510 to Yates); oral patches (e.g., US Pat. Pub. No. 2006/0240087 to Houze et al.); lip balm (e.g., U.S. Pat. No. 7,105,173 to Rolling); dentifrice compositions and toothpicks (e.g., U.S. Pat. No. 5,176,899 to Montgomery; U.S. Pat. No. 5,035,252 to Mondre; U.S. Pat. No. 5,560,379 to Pieczenik; and US Pat. Pub. Nos. 2004/0025900 to Sampson; 2005/0058609 to Nazeri and 2006/0162732 to Winn); and other forms (e.g., U.S. Pat. No. 5,048,544 to Mascarelli; U.S. Pat. No. 6,082,368 to Brown; U.S. Pat. No. 6,319,510 to Yates and U.S. Pat. No. 6,949,264 to McGrew et al.; and US Pat. Pub. Nos. 2005/0008735 to Pearce), which are incorporated herein by reference.

It would be desirable to provide a system capable of delivering or administering at least one active ingredient for therapeutic purposes, particularly by way of oral administration. For example, it would be desirable to provide a therapeutic system possessing both a formulation incorporating at least one active ingredient (e.g., nicotine) and a desirable structure or shape (e.g., a rod-like piece).

### SUMMARY OF THE INVENTION

In one aspect, the present invention relates to a system capable of delivering or administering an active ingredient for therapeutic purposes. Representative active ingredients are those that can be characterized as having a pharmacological effect, and that can be used for therapeutic purposes. Representative active ingredients can be nicotinic compounds (e.g., nicotine-containing compositions, and can include nicotine salt or nicotine polacrilex compositions) intended to be employed for therapeutic purposes (e.g., nicotinic receptor antagonists or nicotinic receptor agonists). At least a portion of the disclosed system has a pharmaceutically acceptable form (e.g., as a drug or as a dietary supplement), and most preferably is adapted for oral delivery.

In another aspect, the present invention relates to a system that incorporates an orally ingestible lozenge and a substrate in contact with the lozenge. The substrate is configured so that a portion thereof is inserted into the mouth of the user, along with the lozenge. The substrate also is configured to allow for passage of drawn air into the mouth of the user. As such, the user of the system ingests the active ingredient(s) of the lozenge and experiences the physiological effects provided by an appropriately designed substrate.

More particularly, the present invention relates to a system for the administration of a therapeutic composition that includes a substrate portion having an upstream end and a downstream end, the upstream end allowing for passage of drawn atmospheric air into the substrate and the downstream end adapted for positioning into user's mouth for draw upon the substrate and inhalation of atmospheric air by the user. The system also includes a lozenge portion incorporating a source of active ingredient in a pharmaceutically acceptable form, the lozenge portion providing for oral ingestion of the active ingredient. The lozenge portion and the substrate portion are physically separate from one another but in contact with each other, with the lozenge being positioned at the downstream end of the substrate. Additionally, the lozenge and substrate portions are positioned relative to one another so that the lozenge portion and a portion of the substrate portion can be located in the user's mouth during use, to provide for delivery of active ingredient from the lozenge and drawn air through the substrate.

Accordingly, in certain aspects, the disclosed system can be described as a system for the administration of a therapeutic composition, the system comprising: a substrate portion having an upstream end and a downstream end, the upstream end allowing for passage of drawn atmospheric air into the substrate and the downstream end adapted for positioning into a user's mouth for draw upon the substrate and inhalation of atmospheric air by the user, a lozenge portion incorporating a source of active ingredient in a pharmaceutically acceptable form, the lozenge portion providing for oral ingestion of the active ingredient, the lozenge portion and the substrate portion being physically separate from one another but in contact with each other, the lozenge being positioned at the downstream end of the substrate, and the lozenge and substrate portions being positioned so that the lozenge portion and a portion of the substrate portion can be located in the user's mouth during use, to provide for delivery of active ingredient from the lozenge and drawn air through the substrate.

The active ingredient can be, for example, a nicotinic compound (e.g., including, but not limited to, compounds selected from the group consisting of nicotine in free base form, salt font, complexed form, and solvated form).

In some embodiments, the substrate portion is non-ingestible. The substrate portion can have varying forms. In some embodiments, the substrate is has a length from upstream end to downstream end of about 60 mm to about 110 mm. In some embodiments, the substrate portion is tubular and has a cross-sectional diameter of about 5 mm to about 10 mm. In some embodiments, the substrate has the form of a hollow tube or in the form of a rod comprising an air permeable material, wherein the rod can optionally be wrapped in a longitudinally-extending circumscribing paper wrap. The air permeable material, where present, can in some embodiments comprise cellulose acetate tow or gathered non-woven polypropylene web. In other embodiments, the substrate portion can have the form of a hollow tube having a plug of air permeable material disposed therein. Such air permeable material can comprise, for example, non-woven cellulose acetate fiber, cotton fibers, or open-cell foam. The substrate portion can, in certain embodiments, further comprise an active ingredient incorporated therein.

The size and shape of the lozenge portion can vary. For example, in some embodiments, the lozenge portion has a longitudinally extending length of about 4 mm to about 11 mm. The lozenge portion can, in certain embodiments, have a longitudinally extending length that is less than about 25 percent of the total length of the substrate. An exemplary but not limiting volume with respect to the lozenge portion is about 500 mm³ to about 2000 mm³. The lozenge can, in some embodiments, have a generally cylindrical shape possessing a passageway therethrough.

The configuration of the substrate portion and the lozenge portion with respect to one another can vary. In some embodiments, the lozenge portion is positioned such that the downstream end of the lozenge portion is positioned downstream of the extreme downstream end of the substrate portion. In some embodiments, the lozenge portion is positioned such that both ends of the lozenge portion are positioned upstream of the extreme downstream end of the substrate portion. In some embodiments, the lozenge portion is positioned such that the downstream end of the lozenge portion is aligned with the extreme downstream end of the substrate portion. In certain embodiments, the lozenge portion and substrate portion are in intimate contact. The lozenge portion and substrate portion can, in some embodiments, be maintained in contact by friction fit between a surface of the lozenge portion and a surface of the substrate portion.

In another aspect of the present disclosure is provided a method of treating or delaying the progression of a condition, disease, or disorder responsive to activation of nicotinic acetylcholinergic receptors in a human subject, comprising administering a therapeutically effective amount of active ingredient in the form of any of the systems disclosed herein to said human subject. Exemplary conditions that can be treated using the compositions of the present invention are dependent upon the active ingredient that is employed. For example, active ingredients that are characterized as nicotinic compounds can be used to treat a wide variety of diseases and disorders, including various diseases and disorders affecting the central nervous system. Additionally, the compositions incorporating nicotinic compounds (e.g., nicotine) can be used as a smoking cessation aids.

### BRIEF DESCRIPTION OF THE DRAWINGS

Having thus described the disclosure in the foregoing general terms, reference will now be made to the accompanying drawings, which are not necessarily drawn to scale, and wherein:

FIG. 1 through FIG. 10 each illustrate a longitudinally extending, side cross-sectional view of a representative system, showing a substrate portion, a lozenge portion and a passageway for drawn air through the lozenge and substrate portions; and

FIG. 11 through FIG. 13 each illustrate an end cross-sectional view of a representative system, showing an inner substrate portion, an outer lozenge portion and a passageway for drawn air through the lozenge and substrate portions.

### DETAILED DESCRIPTION OF THE PREFERRED EMBODIMENTS

The present inventions now will be described more fully hereinafter. The invention may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise.

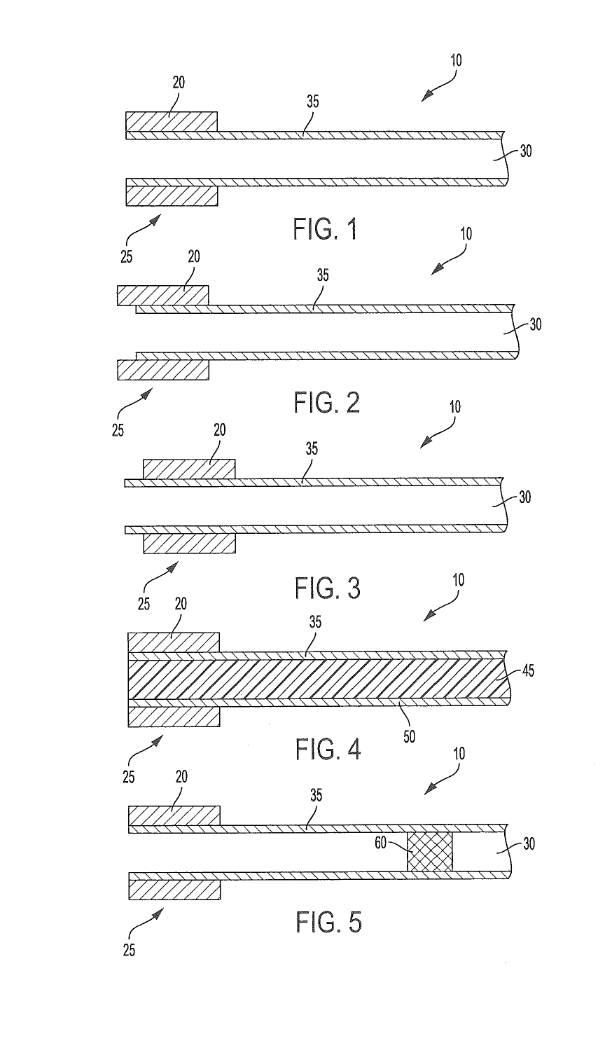

Referring to FIG. 1, there is shown a system 10 that is representative of an embodiment of the present invention. The representative system 10 can be characterized as providing a nicotine replacement therapy type of product. As such, the system includes a lozenge-type formulation 20 (e.g., a NRT type of formulation, such as a formulation incorporating an active ingredient composed of nicotine and suitable excipients) as one of the components of the system, which is located at one extreme end (i.e., the mouth end 25) thereof. The lozenge portion 20 of the representative system is a part or piece of pre-determined shape, and for a representative embodiment, the lozenge portion has a generally cylindrical shape, possessing a passageway 30 extending through. Within the passageway 30 is positioned a portion of the substrate 35, which is a second component of the system. The substrate 35 is a part or piece, separate from the lozenge 20, that for the representative system has a pre-determined shape (e.g., the form of a resilient, hollow, tubular piece). Each end of the tubular piece is open in order to allow for passage of drawn air therethrough. The tubular piece 35 extends through the cylindrical lozenge 20 such that the inner longitudinally-extending surface of the lozenge is in contact with the outer longitudinally-extending surface of the tubular piece. As such, the lozenge 20 is held in place with the tubular piece 35 at the mouth end 25 of the system 10 (e.g., by friction fit and/or through the use of a suitable adhesive or bonding agent). For the representative system, the extreme mouth end regions of each of the lozenge 20 and the substrate 35 are such that they are each in alignment with one another.

A representative generally tubular substrate 35 for the system 10 has dimensions that can vary. For example, the overall length of a representative substrate can range from about 60 mm to about 110 mm, often about 70 mm to about 100 mm, and frequently about 75 mm to about 85 mm. A representative substrate having a generally circular cross-sectional shape can have an outmost diameter of about 5 mm to about 10 mm, and frequently about 7 mm to about 9 mm. The thickness of the hollow tubular substrate can vary; and a representative substrate can be formed from a material having a thickness of about 0.5 mm to about 1.5 mm, and often about 0.7 mm to about 1.2 mm. Although the cross-sectional dimensions of the substrate have been described with reference to a substrate having a generally circular cross-sectional shape, the substrate can have other types of cross-sectional shapes of comparable dimension; for example, cross-sectional shapes that can be characterized as being generally oval, triangular, square, pentagonal, hexagonal or octagonal. Typically, the outer shape of the substrate is comparable to the shape of the inner surface of the lozenge, in order that there is a good fit between the two components, and so that the structural integrity of the system is maintained.

A representative substrate 35 for the system 10 can be constructed from a variety of materials. Representative substrates can be constructed from plastic materials (e.g., extruded plastic materials, such as polypropylene, polystyrene, high density polyethylene, low density polyethylene, or the like), paperboard, such as spiral wound paperboard that has been hardened by coatings or laminates (e.g., such as can be provided with latex-type styrene butadiene coatings), papers, such as those treated with films or coatings that provide moisture resistance and resiliency to the paper stock that is treated (e.g., such as can be provided with ethylene vinylacetate wax types of coatings), or the like. Representative tubular substrates can be obtained by suitable modification of those types of products that have been commercially available under the tradenames 100% Biodegradable Paper Straws by Secret Life, Sip-Stirs by Soodhalter Plastics, Flexible Straws by International Giftware Corporation and Straw Sipper Stirrer by Generation Consumer.

If desired, the substrate 35 can be adapted to incorporate graphics or indicia, and can be provided in a variety of colors or combination of colors; with the visual appearance being a matter of design choice. Additionally, the substrate can be adapted to have a texture that is smooth, rough or patterned; so as to provide desired feel or tactile attributes when grasped by the hand, or held in the mouth, of the user. If desired, the substrate can be composed of paper, or wrapped in paper, so as to mimic many of the visual and tactile attributes of a cigarette. Representative types of representative cigarette paper wrappers and cigarette mouth end tipping papers are set forth in U.S. Pat. No. 5,220,930 to Gentry and U.S. Pat. No. 7,789,089 to Dube et al. and US Pat. Pub. No. 2010/0108081 to Joyce et al., which are incorporated herein by reference. For representative embodiments that are not shown, the outer surface that extends the length of the substrate (or a portion of the length of the substrate) can be overwrapped by cigarette-type paper or tipping-type paper).

The shape, size and dimensions of the lozenge portion 20 can vary. Preferred lozenges have a size so as to be readily placed in the mouth of the user and orally ingested. That is, a lozenge incorporating an active ingredient, such as nicotine, can be used to provide administration of a pharmaceutically effective amount of active ingredient as a result of the oral ingestion of that lozenge. Representative lozenges typically have longitudinally extending lengths of greater than about 4 mm, often greater than about 5 mm and frequently greater than about 6 mm; while such longitudinally extending lengths typically are less than about 15 mm, often less than about 13 mm and frequently less than about 11 mm. Typically, the lozenge portion extends along the length of the substrate portion in an amount that is less than about 25 percent of the total length of the substrate.

The volume of lozenge material or formulation within each lozenge portion 20 can vary. The volume of lozenge material within a representative lozenge typically can be greater that about 500 mm³, often greater than about 600 mm³ and frequently greater than about 700 mm³; while the volume of lozenge material within a representative lozenge typically can be less than about 2000 mm³, often less than about 1500 mm³ and frequently less than about 1200 mm³.

The lozenge 20 and substrate 35 are characterized as being physically separate from one another within the system 10. As such, those components are formulated or constructed from materials that are different in overall composition from one another (e.g., in one embodiment, the lozenge is composed of an orally ingestible lozenge formulation for administration of active ingredient and the substrate is a non-ingestible plastic tube). Additionally, those components most preferably are considered to be in contact, so that the components combine to form such a system that can be considered to be a unitary piece of components that are secured thinly together, but that can be separated from one another during conditions of use. The components in some embodiments can be in intimate contact. Combination of the components to foun a system can be accomplished by friction fit, or by a suitable adhesive. Suitable ways to adhere the components to one another include application of moisture to contact surfaces, application of a solution of sugar in water to contact surfaces, use of binders (e.g., application of aqueous formulations incorporating sodium alginate, carboxymethyl cellulose, xanthan gum, and the like) at contact surfaces, and other such means.

Referring to FIG. 2, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally cylindrical shape, possessing a passageway 30 extending through. Within the passageway 30 is positioned a substrate 35, that for the representative system has the form of a resilient, hollow, tubular piece. The tubular piece 35 extends through the cylindrical lozenge 20 such that the inner longitudinally-extending surface of the lozenge is in contact with the outer longitudinally-extending surface of the tubular piece. For the representative system, the extreme mouth end region of the lozenge 20 extends beyond the extreme mouth end region of the substrate 35 (i.e., the extreme mouth end region of the lozenge is positioned downstream of the extreme mouth end of the substrate). For example, depending upon factors such as the size and physical properties of the lozenge, the extreme mouth end region of the lozenge is positioned up to about 3 mm, often up to about 2 mm downstream from the extreme mouth end region of the substrate; while it is typical for the extreme mouth end region of the lozenge to be positioned at least about 0.5 mm, often at least about 1 mm, downstream from the extreme mouth end region of the substrate.

Referring to FIG. 3, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally cylindrical shape, possessing a passageway 30 extending through. Within the passageway 30 is positioned a substrate 35, that for the representative system has the form of a resilient, hollow, tubular piece. The tubular piece 35 extends through the cylindrical lozenge 20 such that the inner longitudinally-extending surface of the lozenge is in contact with the outer longitudinally-extending surface of the tubular piece. For the representative system, the extreme mouth end region of the substrate 35 extends beyond the extreme mouth end region of the lozenge 20 (i.e., the extreme mouth end region of the lozenge is positioned slightly upstream from the mouth end of substrate). For example, the extreme mouth end region of the lozenge is positioned up to about 6 mm, often up to about 5 mm upstream from the extreme mouth end region of the substrate; while it is typical for the extreme mouth end region of the lozenge to be positioned at least about 1 mm, often at least about 2 mm, upstream from the extreme mouth end region of the substrate.

Referring to FIG. 4, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally cylindrical shape. Within the passageway 30 of the cylindrical lozenge is positioned a substrate 35, that for the representative system has the form of a rod composed of air permeable material 45 (e.g., cellulose acetate tow or gathered non-woven polypropylene web) wrapped in a longitudinally-extending circumscribing paper wrap 50. Each end of the rod is open so as to allow the passage of drawn air therethrough (i.e., the mouth end or downstream end of the rod is placed in the mouth of the user and sucked upon, and atmospheric air entering the upstream end of the rod passes through the rod into the mouth of the user). The air permeable material 45 can be selected and configured so as to allow a desirable draw resistance; and the permeable material can also act as a carrier for ingredients, such as active ingredient and/or volatile flavoring. As such, there is provided a means by which the sensations experienced by the user can be controlled during inhalation when the substrate is drawn upon. The rod 35 extends through the cylindrical lozenge 20 such that the inner longitudinally-extending surface of the lozenge is in contact with the outer longitudinally-extending surface of the rod. As such, the lozenge 20 is held in place with the rod 35 at the mouth end 25 of the system 10. For the representative system, the extreme mouth end regions 25 of each of the lozenge 20 and the substrate 35 are such that they are each in alignment with one another. Alternatively, the extreme mouth end region of the system can have a configuration of the type described with reference to FIG. 2 and FIG. 3. For certain such embodiments, active ingredient can be ingested by having the lozenge dissolve in the user's mouth, as well as by inhalation of active ingredient from the substrate. The predominant administration of the active ingredient preferably is through ingestion of the lozenge, as opposed to active ingredient administered through inhalation; although alternatively, through active ingredient administered through inhalation, as opposed to administration of active ingredient by way of ingestion of lozenge.

The components of the rod-shaped substrate 35, and the construction of that substrate, can vary. A representative type of rod can be provided and constructed (with suitable modifications, if desired) using the types of materials, format and configuration of those types of product that have been marketed commercially under the tradenames “Light-Free” by UltraTech Corp. and “Smoker's Option” by Smokers Options Co. Representative types of permeable materials and overwrap materials, general types of rod configurations and methods suitable for the production of representative types of rod-shaped substrates (including those types of rods incorporating breakable flavor capsules) are set forth in U.S. Pat. No. 4,807,809 to Pryor et al.; U.S. Pat. No. 5,387,285 to Rivers; U.S. Pat. No. 7,740,019 to Nelson et al.; U.S. Pat. No. 7,972,254 to Dube et al.; U.S. Pat. No. 7,984,719 to Dube et al. and U.S. Pat. No. 9,028,385 to Thomas et al.; and US Pat. Pub. No. 2011/0271968 to Carpenter et al., which are incorporated herein by reference. If a rod incorporates breakable flavor capsules, those capsules preferably are positioned upstream from the location of the lozenge portion. Additionally, breakable capsules can be adapted to incorporate active ingredient. Additionally, if desired, the substrate can be provided by a plasticized cellulose acetate tube, such as the so-called “non-wrapped acetate” or “NWA” filter tubes that have traditionally been used as cigarette filter components. Use of suitably adapted rod-shaped substrates can provide for the construction of systems that mimic in many regards the sensations provided by cigarette smoking; for example, suitably rods can be designed to resemble the overall appearance, shape, size, weight, feel, draw characteristics, and the like of a traditional type of cigarette.

Referring to FIG. 5, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally cylindrical shape, possessing a passageway 30 extending therethrough. The tubular piece 35 extends through the cylindrical lozenge 20 such that the inner longitudinally-extending surface of the lozenge is in contact with the outer longitudinally-extending surface of the tubular piece. As such, the lozenge 20 is held in place with the tubular piece 35 at the mouth end 25 of the system 10. For the representative system, the extreme mouth end regions of each of the lozenge 20 and the substrate 35 are such that they are each in alignment with one another. Within a portion of length of the tubular piece 35 is positioned a plug of air permeable material 60 (e.g., non-woven cellulose acetate fiber, sponge-type material, cotton fibers, open-cell rubber-type foam, or the like). The air permeable material 60 provides for passage of drawn air therethrough. Additionally, the air permeable material can be selected and configured so as to allow a desirable draw resistance; and the permeable material can act as a carrier for ingredients, such as active ingredient and/or volatile flavoring. A typically plug, has a longitudinally extending length of about 5 mm to about 25 mm, often about 10 mm to about 20 mm. Although the positioning of the plug 60 along the length of the tubular piece 35 can vary, it is preferable to position that plug towards the extreme upstream end of the system 10. A representative tubular piece 35 having a plug of air permeable material 60 positioned therein can be provided and constructed (with suitable modifications, if desired) using the types of materials, format and configuration of a product that has been marketed commercially under the tradename “Favor” by Advanced Tobacco Products, Inc. For the representative system, the extreme mouth end regions of each of the lozenge 20 and the substrate 35 are such that they are each in alignment with one another. Alternatively, the extreme mouth end region of the system can have a configuration of the type described with reference to FIG. 2 and FIG. 3. For certain such embodiments, active ingredient can be ingested by having the lozenge dissolve in the user's mouth, as well as by inhalation of active ingredient from the substrate. Preferably, the predominant administration of the active ingredient is through ingestion of the lozenge, as opposed to active ingredient administered through inhalation.

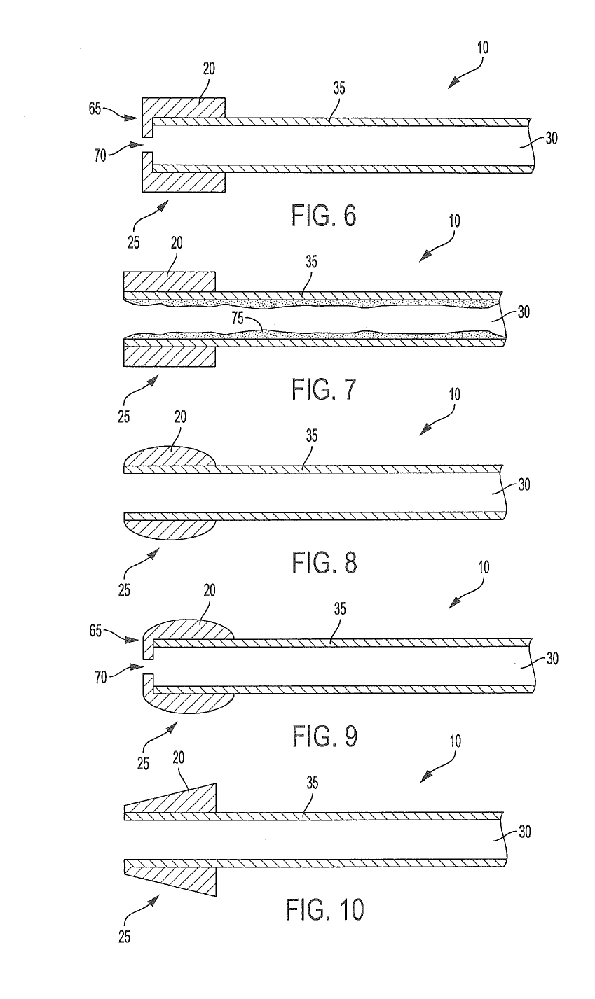

Referring to FIG. 6, there is shown a representative system 10 that is similar in many regards to that system described with reference to FIG. 2. The system 10 also includes a lozenge-type formulation 20 located at one extreme end (i.e., the mouth end 25) thereof. The lozenge portion 20 of the representative system has a generally cylindrical shape, possessing a passageway 30 extending through. Within the passageway 30 is positioned a substrate 35, that for the representative system has the form of a resilient, hollow, tubular piece. The tubular piece 35 extends through the generally cylindrical lozenge 20 such that the inner longitudinally-extending surface of the lozenge is in contact with the outer longitudinally-extending surface of the tubular piece. For the representative system, the extreme mouth end region of the lozenge 20 extends beyond the extreme mouth end region of the substrate 35. In addition, the lozenge 20 is adapted so as to have a type of “cup like” shape, in that the extreme mouth end portion of the lozenge 65 extends over a portion of the extreme mouth end region of the passageway 30. As such, mouth end passageway 70 has a cross-sectional area that is less than that of the cross-sectional area of the air passageway (i.e., the inner cross-sectional area) of the substrate 35.

Typically, the extreme mouth end portion 65 of the lozenge 20 has a longitudinally-extending length of about 2 mm to about 5 mm. The mouth end passageway 70 of the lozenge has a cross-sectional area that is less than that of the air passageway 30 of the substrate; but typically, the cross-sectional area of that mouth end passageway is at least about 20 percent, often at least about 30 percent and sometimes at least about 50 percent of the cross-sectional area of the passageway 30 of the substrate 35.

Referring to FIG. 7, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally cylindrical shape, possessing a passageway 30 extending through. Within the passageway 30 is positioned a substrate 35, that for the representative system has the form of a resilient, hollow, tubular piece. The tubular piece 35 extends through the cylindrical lozenge 20 such that the inner longitudinally-extending surface of the lozenge is in contact with the outer longitudinally-extending surface of the tubular piece. Additionally, at least a portion of the inner longitudinally-extending surface of the substrate 35 is coated (or is otherwise provided with, such as by layering or laminating a flavor and/or aroma infused material) a carrier material 75 that acts as a source of ingredients, such as volatile flavorings. For example, flavoring agents and aromatic ingredients can be incorporated within carrier materials, or can be infused within carriers, films or coatings (e.g., such as those films or coatings that can be provided using starches, ethylene vinylacetate, sodium carboxymethyl cellulose, waxes, ethyl cellulose, polypropylene, or the like). See also, for example, those types of representative technologies set forth in U.S. Pat. No. 6,706,120 to Miyauchi et al. and U.S. Pat. No. 7,381,277 to Gonterman et al., which are incorporated herein by reference. For the representative system, the extreme mouth end regions of each of the lozenge 20 and the substrate 35 are such that they are each in alignment with one another. Alternatively, the extreme mouth end region of the system can have a configuration of the type described with reference to FIG. 2 and FIG. 3.

Referring to FIG. 8, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally ring-like shape, possessing a passageway 30 extending through. That is, the lozenge has outer cross-sectional dimensions that vary along the longitudinal length of the system 10. For the embodiment shown, the cross-sectional dimensions toward each end of the lozenge 20 are relatively small, while the cross-sectional dimensions toward the middle of the lozenge are relatively large. Within the passageway 30 is positioned a substrate 35, that for the representative system has the form of a resilient, hollow, tubular piece. The tubular piece 35 extends through the cylindrical lozenge 20 such that the inner longitudinally-extending surface of the lozenge is in contact with the outer longitudinally-extending surface of the tubular piece. For the representative system, the extreme mouth end regions of each of the lozenge 20 and the substrate 35 are such that they are each in alignment with one another. Alternatively, the extreme mouth end region of the system can have a configuration of the type described with reference to FIG. 2 and FIG. 3.

Referring to FIG. 9, there is shown a representative system 10 that is similar in many regards to that system described with reference to FIG. 6. The system 10 also includes a lozenge-type formulation 20 located at one extreme end (i.e., the mouth end 25) thereof. The lozenge portion 20 of the representative system has a generally ring-like shape (e.g., generally of the type described with reference to FIG. 8), possessing a passageway 30 extending through. Within the passageway 30 is positioned a substrate 35, that for the representative system has the form of a resilient, hollow, tubular piece. The tubular piece 35 extends through the lozenge 20 such that the inner longitudinally-extending surface of the lozenge is in contact with the outer longitudinally-extending surface of the tubular piece. For the representative system, the extreme mouth end region of the lozenge 20 extends beyond the extreme mouth end region of the substrate 35. In addition, the lozenge 20 is adapted so as to have a type of “cup like” shape, in that the extreme mouth end portion of the lozenge 65 extends over a portion of the passageway 30. As such, mouth end passageway 70 has cross-sectional dimensions that are less than that of the cross-sectional dimensions of the air passageway (i.e., the inner cross-sectional diameter) of the substrate 35. For example, the dimensions of mouth end passageway of the lozenge often can be about 20 percent to about 90 percent, often about 30 to about 80 percent, and frequently about 40 percent to about 70 percent, of the dimensions of the passageway of the substrate.

Referring to FIG. 10, there is shown a representative system 10 that is similar in many regards to that system described with reference to FIG. 1. The system 10 also includes a lozenge-type formulation 20 located at one extreme end (i.e., the mouth end 25) thereof. The lozenge portion 20 of the representative system has a generally frustoconical type of shape, possessing a passageway 30 extending through. That is, the lozenge has outer cross-sectional dimensions that vary along the longitudinal length of the system 10. For the embodiment shown, the cross-sectional dimensions of the lozenge 20 are relatively small at the extreme mouth end 25 region, while the cross-sectional diameter toward the upstream region of the lozenge are relatively large. Within the passageway 30 is positioned a substrate 35, that for the representative system has the form of a resilient, hollow, tubular piece. The tubular piece 35 extends through the cylindrical lozenge 20 such that the inner longitudinally-extending surface of the lozenge is in contact with the outer longitudinally-extending surface of the tubular piece. For the representative system, the extreme mouth end regions of each of the lozenge 20 and the substrate 35 are such that they are each in alignment with one another. Alternatively, the extreme mouth end region of the system can have a configuration of the type described with reference to FIG. 2 or FIG. 3. Alternatively, for a similar embodiment (not shown) the cross-sectional dimensions of the lozenge can be such that the cross-sectional dimensions of the lozenge 20 are relatively large at the extreme mouth end 25 region, while the cross-sectional dimensions toward the upstream region of the lozenge are relatively small. Additionally, for a similar embodiment (not shown) the cross-sectional dimensions of the lozenge can be such that the cross-section dimensions of the lozenge are relatively large at the extreme mouth end region, relatively large at the extreme upstream region, and relatively small in the central region.

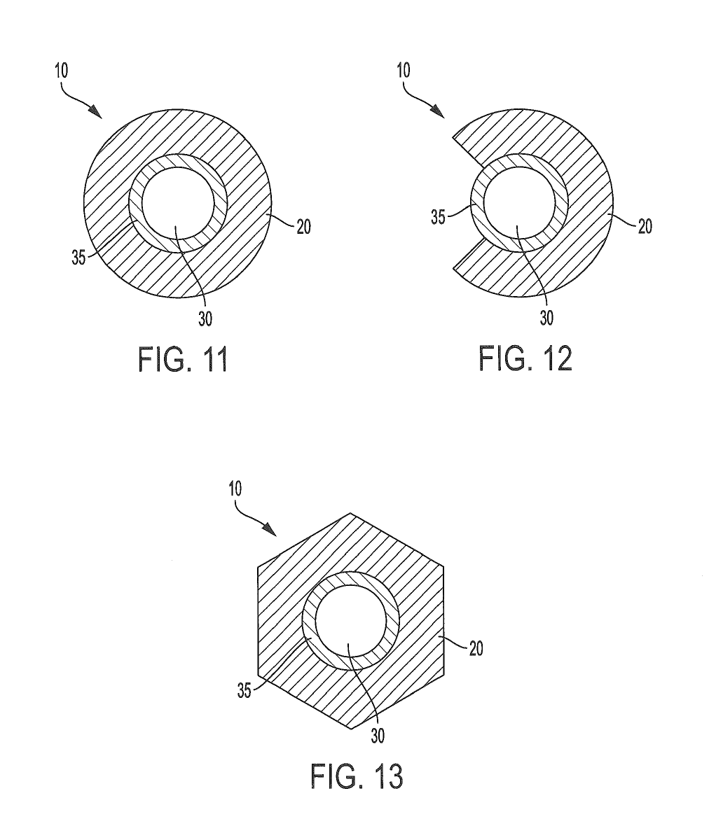

Referring to FIG. 11, there is shown an end view of a representative preferred system 10, such as the representative system described with reference to FIG. 1. The lozenge 20 encircles the tubular substrate 35, while providing an air passageway 30. For the embodiment shown, the lozenge 20 has an outer cross-sectional shape that can be considered to be generally circular in nature.

Referring to FIG. 12, there is shown an end view of a representative preferred system 10, such as the representative system described with reference to FIG. 1. For the embodiment shown, the lozenge 20 partially encircles the tubular substrate 35, while providing an air passageway 30. Most preferably, the lozenge portion encircles at least half of the circumference of the substrate. Most preferably, the lozenge encircles at least half of the circumferential surface of the substrate.

Referring to FIG. 13, there is shown an end view of a further representative system 10, such as the representative system described with reference to FIG. 1. The lozenge 20 encircles the tubular substrate 35, while providing an air passageway 30 therethrough. For the embodiment shown, the inner passageway has a generally circular cross-section shape; and the lozenge 20 has an outer cross-sectional shape that can be considered to be generally hexagonal in nature. For embodiments not shown, the lozenge 20 can have a variety of other outer cross-sectional shapes, such as oval, triangular, square, pentagonal or octagonal.

In use, an embodiment of a representative system 10 is grasped by the user by the substrate 35. The mouth end 25 of the system 10 is inserted into the mouth of the user. As such, the user orally administers the lozenge portion 20 of the system. The lozenge portion, which now is located within the mouth of the user, then is treated in a manner so that the components of the lozenge are orally ingested (e.g., the lozenge is chewed, licked or sucked upon, so that lozenge is subdivided into ingestible pieces or dissolved). As such, the active ingredient (which is contained within the lozenge portion) is administered to the user through his/her mouth, and the user experiences organoleptic, trigeminal, flavor attributes and other sensations associated with that lozenge being placed in the mouth of the user. In addition, the substrate 35 can be grasped by the user with his/her fingers (typically in a region of the substrate upstream from the lozenge), moved around mouth area of the use, manipulated and moved by the lips of the user, or otherwise manipulated by the user (e.g., to generally simulate the types of actions performed by a smoker smoking a cigarette). Furthermore, the user can draw air through the substrate 35, inhale volatile components positioned with in the substrate, or otherwise manipulate the substrate (e.g., to generally simulate the types of actions performed by a smoker inhaling smoke while smoking a cigarette). When the lozenge 20 has been ingested, the user may continue to manipulate the substrate 35, and then dispose of that substrate. As such, components of the system 10 typically are administered in a form adapted for buccal or sublingual delivery. For example, nicotine-containing compositions can be administered using the manners and methods typically used for the administration of traditional types of nicotine-containing lozenges, and the substrate is employed so that other physical actions associated with cigarette smoking can be mimicked to some degree. As such, the user experiences various sensations (e.g., trigeminal, organoleptic and pharmacological effects provided by the active ingredient, as well as those ingredients that provide aroma and flavor) and the physiological effects (e.g., those provided by the feel and movement provided by the substrate, as well as the sensations provided by inhalation of air through the substrate).

For each system unit, active ingredient is incorporated within a formulation that is considered to have a form that can be characterized as a lozenge. As such, representative formulations can have the components, and can be formulated, as have traditional products that have been characterized as lozenge, pill, capsule, caplet, mini lozenge, tablet, microtab, or other tablet-type products. A representative lozenge can be considered to have an overall shape that can be considered to be generally spherical, generally cylindrical, helical, obloid, square, rectangular, or the like. See, for example, the types of nicotine-containing lozenges, lozenge formulations, lozenge formats and configurations, lozenge characteristics and techniques for formulating or manufacturing lozenges set forth in U.S. Pat. No. 4,967,773 to Shaw; U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al.; U.S. Pat. No. 6,248,760 to Wilhelmsen and U.S. Pat. No. 7,374,779 to Chen et al.; US Pat. Pub. Nos. 2001/0016593 to Wilhelmsen; 2004/0101543 to Liu et al.; 2006/0120974 to Mcneight; 2008/0020050 to Chau et al.; 2009/0081291 to Gin et al. and 2010/0004294 to Axelsson et al.; and PCT WO 91/09599 to Carlsson et al., which are incorporated herein by reference. Representative formulations for suitable types of lozenges also are set forth US Pat. Pub. Nos. 2013/0209540 to Duggins et al and 2013/078307 to Holton et al., which are incorporated herein by reference. See, also, the types of nicotine-containing formulations that can be produced using those teachings set forth, or suitably modifying the teachings set forth, in US Pat. Nos. 2012/0138073 to Cantrell et al.; 2012/0138074 to Cantrell et al.; 2013/0274296 to Jackson et al. and 2015/0101627 to Marshall et al.; and U.S. patent application Ser. No. 14/180,710 to Lamp et al. The general nature of a representative composition can be such that the resulting lozenge is soft or hard to the touch, or of intermediate softness or hardness; and as such, the composition can be considered to be malleable, flexible, chewy, resilient, brittle, or the like.

Representative types of lozenges can be formulated using the types of ingredients incorporated within those lozenges that have been commercially available as Commit Nicotine Polacrilex Lozenge 2 mg by GlaxoSmithKline Consumer Healthcare, LP and Nicotine Lozenge, Nicotine Polacrilex Lozenge, 2 mg (nicotine) by Rite Aid Pharmacy.

Formulations of representative lozenges may include short-term, rapid-onset, rapid-offset, controlled release, sustained release, delayed release, and pulsatile release formulations, providing the formulations achieve administration of the active ingredient. See, also, Remington's Pharmaceutical Sciences, 18^{th} ed.; Mack Publishing Company, Eaton, Pa., (1990), which is incorporated herein by reference.

Lozenges can be formulated so as to provide a delayed release of the active ingredient (i.e., the nicotinic compound), such as by application of a coating. Delayed release coatings are known in the art, and dosage forms containing such may be prepared by any known suitable method. Such methods generally involve application of a delayed release coating composition after preparation of the solid dosage form (e.g., a tablet or caplet). Application of the coating can be by methods such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms also can be formulated to provided sustained release (i.e., releasing the active ingredient over a prolonged period of time), and may or may not also be delayed release. Sustained release formulations are known in the art and are generally prepared by dispersing the active ingredient within a matrix of a gradually degradable or hydrolyzable material, such as an insoluble plastic, a hydrophilic polymer, or a fatty compound. Alternatively, a solid dosage form may be coated with such degradable or hydrolysable material.

As a result, for compositions ingested by insertion in the mouth of the human subject, the release rate of active ingredient during use of the product can vary from relatively fast to relatively slow, depending upon factors such as the design of the product and the use of product by the subject using that product. See also, by way of example, the types of products proposed and the types of active ingredient release disclosed in U.S. Pat. No. 4,655,231 to Ray et al.; U.S. Pat. No. 5,147,654 to Place et al.; U.S. Pat. No. 5,543,424 to Carlsson et al.; U.S. Pat. No. 6,268,386 to Thompson; U.S. Pat. No. 6,319,510 to Yates; U.S. Pat. No. 6,488,953 Halliday et al.; U.S. Pat. No. 6,709,671 to Zerbe et al.; U.S. Pat. No. 7,025,983 to Leung et al.; U.S. Pat. No. 7,105,173 to Rolling; U.S. Pat. No. 7,115,297 to Stillman; U.S. Pat. No. 7,435,749 to Knight and U.S. Pat. No. 7,491,406 to Leung et al.; and US Pat. Pub. Nos. 2004/0191322 to Hansson; 2006/0198873 to Chan et al.; 2006/0240087 to Houze et al.; 2006/0204559 to Bess et al.; 2007/0269492 to Steen et al.; 2008/0020050 to Chau et al.; 2008/0286340 to Andersson et al.; 2008/0292683 to Sanghvi et al. and 2009/0004248 to Bunick et al., which are incorporated herein by reference.

The present invention involves providing a composition that can be used for therapeutic purposes. That is, the composition can be used to treat the cause or symptoms associated with a disease or ailment, or otherwise provide for the well-being of the subject to which the composition is administered. The composition can be used as a pharmaceutical composition or as a dietary supplement. The composition incorporates at least one active ingredient, and the composition can be suitably adapted for nasal or oral delivery of that active ingredient. One particularly preferred active ingredient is a compound that can be characterized as a nicotinic compound. Various nicotinic compounds, and methods for their administration, are set forth in US Pat. Pub, No. 2011/0274628 to Borschke, which is incorporated herein by reference. As used herein, “nicotinic compound” or “source of nicotine” often refers to naturally-occurring or synthetic nicotinic compound unbound from a plant material, meaning the compound is at least partially purified and not contained within a plant structure, such as a tobacco leaf Most preferably, nicotine is naturally-occurring and obtained as an extract from a Nicotiana species (e.g., tobacco). The nicotine can have the enantiomeric foam S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis. Despite the fact that nicotine can be extracted from Nicotiana species, it is highly preferred that the nicotine (and the composition and products produced in accordance with the present invention) are virtually or essentially absent of other components obtained from or derived from tobacco.

The weight of ingredients within each piece or unit of lozenge type of product can vary. For example, a representative lozenge generally weighs at least about 100 mg, often at least about 200 mg, and frequently at least about 300 mg; while the weight of a representative unit for such products generally does not exceed about 1.5 g, often does not exceed about 1 g, and frequently does not exceed about 0.75 g.

Nicotinic compounds can include nicotine in free base form, salt form, as a complex, or as a solvate. See, for example, the discussion of nicotine in free base form in US Pat. Pub. No. 2004/0191322 to Hansson, which is incorporated herein by reference. At least a portion of the nicotinic compound can be employed in the form of a resin complex of nicotine, where nicotine is bound in an ion exchange resin, such as nicotine polacrilex. See, for example, U.S. Pat. No. 3,901,248 to Lichtneckert et al., which is incorporated herein by reference. At least a portion of the nicotine can be employed in the form of a salt. Salts of nicotine can be provided using the types of ingredients and techniques set forth in U.S. Pat. No. 2,033,909 to Cox et al. and U.S. Pat. No. 4,830,028 to Lawson et al., and Perfetti, Beitrage Tabakforschung Int., 12: 43-54 (1983), which are incorporated herein by reference. See, also, US Pat. Pub. No. 2011/01666668809 to Brinkley et al. and U.S. patent application Ser. No. 14/721,283 to Dull et al., filed May 26, 2015, which is incorporated herein by reference. A representative nicotine salt is nicotine bitartrate dihydrate. Additionally, salts of nicotine have been available from sources such as Pfaltz and Bauer, Inc. and K&K Laboratories, Division of ICN Biochemicals, Inc.

In some embodiments, the nicotinic compound can be employed in multiple forms. For example, the nicotine can be employed within the composition as a mixture of at least two salts (e.g., two different organic acid salts, such as a mixture of nicotine bitartrate dihydrate and nicotine levulinate), as at least two salts that are segregated within the composition, in a free base form and salt form, in a free base form and a salt form that are segregated within the composition, in a salt form and in a complexed form (e.g., a resin complex such as nicotine polacrilex), in a salt for and in a complexed form that are segregated with in the composition, in a free base form and a complexed form, in a free base form and a complexed form that are segregated within the composition, or the like. As such, each single dosage unit or piece (e.g., lozenge) can incorporate at least two forms of nicotine. Alternatively, one form of nicotine can be incorporated within the lozenge and another form of nicotine can be incorporated within the substrate.

A nicotinic compound, in particular as compound such as nicotine, also can be employed in combination with other so-called tobacco alkaloids (i.e., alkaloids that have been identified as naturally occurring in tobacco). For example, nicotine, as employed in accordance with the present invention, can be employed in combination with nornicotine, anatabine, anabasine, and the like, and combinations thereof. See, for example, Jacob et al., Am. J. Pub. Health, 5: 731-736 (1999), which is incorporated herein by reference.

The compositions of the invention most preferably possess a form that is pharmaceutically effective and pharmaceutically acceptable. That is, the composition most preferably does not incorporate to any appreciable degree, or does not purposefully incorporate, significant amounts of components of tobacco, other than nicotine. As such, pharmaceutically effective and pharmaceutically acceptable compositions do not include tobacco in parts or pieces, processed tobacco components, or many of the components of tobacco traditionally present within tobacco-containing cigarettes, cigars, pipes, or smokeless forms of tobacco products. Highly preferred compositions that are derived by extracting naturally-occurring nicotine from tobacco include less than 5 weight percent of tobacco components other than nicotine, more often less than about 0.5 weight percent, frequently less than about 0.25 weight percent, and typically are entirely absent or devoid of components of tobacco, processed tobacco components, or components derived from tobacco, other than nicotine, based on the total weight of the composition.

Preferred lozenges incorporate various pharmaceutically acceptable excipients. By “pharmaceutically acceptable excipient” is meant an excipient that can be used to facilitate the storage, administration, and/or the healing effect of an active agent (e.g., a nicotinic compound). The excipients are pharmaceutically acceptable in the sense of being compatible with the other ingredients of the formulation and not unduly deleterious to the recipient thereof; and they may also reduce any undesirable side effects of the active agent. See, Wang et al., J. Parent. Drug Assn., 34(6): 452-462 (1980), which is incorporated herein by reference. Exemplary pharmaceutical excipients suitable for use in the compositions according to the invention are listed in Remington: The Science & Practice of Pharmacy, 21^{st} ed., Lippincott Williams & Wilkins (2006); in the Physician's Desk Reference, 64^{th} ed., Thomson PDR (2010); and in Handbook of Pharmaceutical Excipients, 6^{th} ed., Eds. Raymond C. Rowe et al., Pharmaceutical Press (2009), which are incorporated herein by reference.

The various excipients can vary, and the selection and amount of each excipient can depend upon factors such as the ultimate form and function of product that is desired. See, for example, the types of ingredients, relative amounts and combinations of ingredients, nicotine-containing formulations and preparation processes for nicotine-containing products set forth in U.S. Pat. No. 5,512,306 to Carlsson et al.; U.S. Pat. No. 5,525,351 to Dam; U.S. Pat. No. 5,549,906 to Santus; U.S. Pat. No. 5,711,961 to Reiner et al.; U.S. Pat. No. 5,811,126 to Krishnamurthy; U.S. Pat. No. 5,939,100 to Albrechtsen et al.; U.S. Pat. No. 6,024,981 to Khankari et al.; U.S. Pat. No. 6,083,531 to Humbert-Droz et al.; U.S. Pat. No. 6,090,401 to Gowan, Jr. et al.; U.S. Pat. No. 6,110,495 to Dam; U.S. Pat. No. 6,248,760 to Wilhelmsen; U.S. Pat. No. 6,280,761 to Santus; U.S. Pat. No. 6,426,090 to Ream et al.; U.S. Pat. No. 6,569,463 to Patel et al.; U.S. Pat. No. 6,583,160 to Smith et al.; U.S. Pat. No. 6,585,997 to Moro et al.; U.S. Pat. No. 6,676,959 to Andersson et al.; U.S. Pat. No. 6,893,654 to Pinney et al.; U.S. Pat. No. 7,025,983 to Leung et al. and U.S. Pat. No. 7,163,705 Johnson et al.; US Pat. Pub. Nos. 2003/0176467 to Andersson et al.; 2003/0235617 to Martino et al.; 2004/0096501 to Maya et al.; 2004/0101543 to Liu et al.; 2004/0191322 to Hansson; 2005/0053665 to Ek et al.; 2005/0123502 to Chan et al.; 2008/0038209 to Andersen et al.; 2008/0286341 to Andersson et al.; 2009/0023819 to Axelsson; 2009/0092573 to Andersen; 2010/0004294 to Axelsson et al.; 2010/0061940 to Axelsson et al. and 2013/0098337 to Borschke et al.; and EP 1458388 to Lindell et al., which are incorporated herein by reference.

Representative types of excipients that are particularly useful for the manufacture of nicotine-containing lozenges include fillers or carriers for active ingredients (e.g., calcium polycarbophil, microcrystalline cellulose, cornstarch and modified corn starch, isomalt, maltodextrin, silicon dioxide or calcium carbonate), thickeners, film formers and binders (e.g., hydroxypropyl cellulose, hydroxypropyl methylcellulose, acacia, sodium alginate, xanthan gum, maltitol syrup and gelatin), buffers and pH control agents (e.g., magnesium oxide, magnesium hydroxide, sodium dihydrogen phosphate, sodium citrate, sodium acetate, monopotassium phosphate, potassium carbonate, sodium carbonate, potassium bicarbonate, levulinic acid, sodium bicarbonate, or mixtures thereof), antiadherents (e.g., talc), glidants (e.g., colloidal silica), natural or artificial sweeteners (e.g., saccharin, acesulfame K, aspartame, sucralose, isomalt, lactose, mannitol, sorbitol, xylitol and sucrose), humectants (e.g., glycerin), preservatives and antioxidants (e.g., butylhydroxy toluene, sodium benzoate, benzyl alcohol and ascorbyl palmitate), surfactants (e.g., polysorbate 80), natural or artificial flavors (e.g., eugenol, mint, cinnamon, cherry, menthol or other fruit flavors), dyes or pigments (e.g., titanium dioxide or D&C Yellow No. 10), and lubricants or processing aids (e.g., calcium stearate, magnesium stearate or coconut oil). Certain types of lozenges also can have outer coatings composed of ingredients capable of providing acceptable outer coatings (e.g., an outer coating can be composed of ingredients such as carnauba wax, and pharmaceutically acceptable forms of shellacs, glazing compositions and surface polish agents).

The amount of active ingredient within the system can vary. For example, nicotine can be delivered by oral ingestion of the lozenge, or from a combination of oral ingestion of the lozenge and inhalation from within the substrate. For a typical system, the amount of nicotine within each dosage unit (i.e., combination of lozenge and substrate) generally is at least about 0.5 mg, generally is at least 1 mg, often is at least about 1.5 mg, and frequently is at least about 2 mg; while the amount of nicotine within each dosage unit typically does not exceed about 10 mg, generally does not exceed about 8 mg, often does not exceed about 6 mg, and frequently does not exceed about 5 mg, calculated as nicotine base. Exemplary types of systems can incorporate about 2 mg, about 3 mg, about 4 mg or about 5 mg per lozenge piece, calculated as nicotine base.

The dose of active ingredient (i.e., all the various nicotine forms capable of being delivered by a given system) is that amount effective to treat some symptoms of, or prevent occurrence of the symptoms of, the condition, disease, or disorder from which the subject or patient suffers. By “effective amount”, “therapeutic amount” or “effective dose” is meant that amount sufficient to elicit the desired pharmacological or therapeutic effects, thus resulting in effective prevention or treatment of the condition, disease, or disorder. Thus, an effective amount of active ingredient is an amount sufficient to enter relevant regions of the body (e.g., including passing across the blood-brain barrier of the subject), to bind to relevant receptor sites in the CNS and PNS of the subject, and/or to elicit neuropharmacological effects (e.g., elicit neurotransmitter secretion, thus resulting in effective prevention or treatment of the condition, disease, or disorder). Prevention of the disorder is manifested, for example, by delaying the onset of the symptoms of the condition, disease, or disorder. Treatment of the disorder is manifested by, for example, a decrease in the symptoms associated with the condition, disease, or disorder or an amelioration of the reoccurrence of the symptoms thereof.

For compositions of the present invention, the intended daily dose of the active ingredient can vary. The overall dose of active ingredient can depend upon factors such as the weight of the subject ingesting the composition, the type of condition, disease, or disorder being treated, the state or severity of the condition, disease, or disorder being treated, the desired pharmacological effect, or other such factors. Typically, the amount of nicotine active ingredient, calculated as nicotine base, administered to a subject per day is at least about 2 mg, often is at least about 4 mg, and frequently is at least about 10 mg. Typically, the amount of nicotine active ingredient administered to a subject per day does not exceed about 60 mg, often does not exceed about 50 mg, and frequently does not exceed about 40 mg. See also, for example, the types of dosing regimens and administration techniques set forth in U.S. Pat. No. 5,593,684 to Baker et al. and U.S. Pat. No. 6,660,754 to Kyle et al.; and US Pat. Pub. Nos. 2004/0006113 to Sachs; 2005/0214229 to Pinney et al.; 2008/0124283 to Andersen and 2009/0293895 to Axelsson et al.; which are incorporated herein by reference.

The compositions of the present invention can be used for treatment of a wide variety of conditions, diseases, and disorders responsive to stimulation of one or more types of nicotinic acetylcholinergic receptors (nAChRs). The compositions can be used to treat those types of conditions, diseases, and disorders that have been reported to be treatable through the use or administration of nicotine as an agonist or antagonist of nAChRs. As such, the compositions can be used to treat various CNS conditions, diseases, and disorders, and the compositions also can be used as nicotine-containing products, such as smoking cessation aids (i.e., as components of NRT).

The following examples are illustrative of representative examples of the present invention that can be employed to provide for oral ingestion of nicotine for therapeutic purposes, such as NRT, but the examples should not be construed as limiting the scope of the present invention. Unless otherwise noted, all parts and percentages are by weight.

### Example 1

A formulation for a lozenge that has been distributed commercially as Nicotine Polacrilex Lozenge, 2 mg (Nicotine) by Perrigo is provided. However, rather than providing a generally circular and somewhat cylindrical shaped lozenge of about 17 mm in diameter, a lozenge having an outermost diameter of about 21 mm and a thickness of about 4 mm is provided using similar preparation techniques. Through the center of the lozenge is formed or fashioned a longitudinally-extending passageway having a diameter of about 9 mm Into the passageway of the lozenge is inserted a tubular substrate of the type that has been marketed under the tradename “Favor.” The substrate possesses a porous plug positioned near its extreme upstream end, and the plug acts as a carrier for nicotine. The exact size of the central passageway of the lozenge is such that the lozenge and substrate are maintained securely in position by friction fit. The lozenge is positioned the mouth end region of the substrate so that the extreme mouth end of the lozenge is positioned about in alignment with the extreme mouth end of the substrate. As such, there is provided a system having many of the attributes of the system that has been described with reference to FIG. 5. The mouth end of the substrate is inserted into the user's mouth, and the user ingests the components of the lozenge. Inhalation of air through the substrate provides the many of the sensations associated with drawing upon a cigarette, and nicotine in vapor form also is inhaled into the user's lungs within air drawn through the substrate. As such, the user receives nicotine from two sources, the lozenge and the substrate.

### Example 2

A system generally of the type described with reference to Example 1 is provided. However, the inner face of the passageway through the lozenge is moistened with distilled water prior to assembly; and the outer surface of the mouth end region of the substrate is moistened with distilled water. The system then is constructed by inserting the mouth end of the substrate through the passageway of the lozenge so that the extreme mouth end of the lozenge is positioned about 4 mm upstream from the extreme mouth end of the substrate. The resulting system then is dried by subjecting the system to a stream of dry air. The use of moisture and the drying of the system act to provide a binding or bonding of the lozenge to the substrate. As such, there is provided a system having good physical integrity.

### Example 3

A lozenge generally of the type described with reference to Example 1 is provided, except that the longitudinally-extending passageway through the lozenge has a diameter of about 6 mm and the overall diameter of the lozenge is about 20 mm. Additionally, the substrate that is selected is a tube having a length of about 80 mm and a diameter of about 6.2 mm. The plastic tube is provided by cutting a plastic straw to the desired length. The straw that is employed to provide the substrate has been commercially distributed as Home 360° Flexible Straws by DZA Brands, LLC. The system then is constructed by inserting one end of the substrate through the passageway of the lozenge so that the extreme mouth end of the lozenge is positioned about 4 mm upstream from the extreme mouth end of the substrate. As such, there is provided a system having many of the attributes of the system that has been described with reference to FIG. 3.

### Example 4

A lozenge generally of the type described with reference to Example 1 is provided, except that the longitudinally-extending passageway through the lozenge has a diameter of about 8 mm. Additionally, the substrate that is selected is generally cylindrical rod composed of fibrous tow wrapping in paper, and that rod has a length of about 79 mm and a diameter of about 8 mm. The rod that is employed to provide the substrate has been commercially distributed as Smoker's Option Menthol. Into passageway of the lozenge is inserted the cylindrical substrate. The exact size of the central passageway of the lozenge is such that the lozenge and substrate are maintained securely in position by friction fit. The lozenge is positioned so that the extreme mouth end of the lozenge is positioned about in alignment with the extreme mouth end of the substrate. As such, there is provided a system having many of the attributes of the system that has been described with reference to FIG. 4.

### Example 5

A system generally of the type described with reference to Example 3 is provided. However, the lozenge that is employed is a generally translucent lozenge composed of about 82 parts isomalt, about 16.2 parts maltitol syrup, about 0.4 part nicotine bitartate dihydrate, about 0.65 part sodium carbonate, about 0.01 part sucralose and about 0.65 part flavoring and coloring; and is produced using those types of techniques set forth in US Pat. Pub. No. 2013/0074855 to Holton et al., which is incorporated herein by reference.

### Example 6

A system generally of the type described with reference to Example 5 is provided. However, the translucent lozenge is fashioned so as to have a longitudinally extending length of about 7 mm and an overall diameter of about 15 mm. Through the center of the lozenge is formed or fashioned a longitudinally-extending passageway having a diameter of about 6 mm. Into passageway of the lozenge is inserted a tubular substrate of the type described in Example 3. The exact size of the central passageway of the lozenge is such that the lozenge and substrate are maintained securely in position by friction fit. The lozenge is positioned the mouth end region of the substrate so that the extreme mouth end of the lozenge is positioned about in alignment with the extreme mouth end of the substrate. As such, there is provided a system having many of the attributes of the system that has been described with reference to FIG. 1.

What is claimed is:

1. A system for the administration of a therapeutic composition, the system comprising:
a substrate portion having an upstream end and a downstream end, the upstream end allowing for passage of drawn atmospheric air into the substrate and the downstream end adapted for positioning into a user's mouth for draw upon the substrate and inhalation of atmospheric air by the user,
a lozenge portion incorporating a source of active ingredient in a pharmaceutically acceptable form, the lozenge portion providing for oral ingestion of the active ingredient,
the lozenge portion and the substrate portion being physically separate from one another but in contact with each other, the lozenge being positioned at the downstream end of the substrate, and
the lozenge and substrate portions being positioned so that the lozenge portion and a portion of the substrate portion can be located in the user's mouth during use, to provide for delivery of active ingredient from the lozenge and drawn air through the substrate.

2. The system of claim 1, wherein the active ingredient is a nicotinic compound.

3. The system of claim 2, wherein the nicotinic compound is selected from the group consisting of nicotine in free base form, salt form, complexed form, and solvated form.

4. The system of claim 1, wherein the substrate portion is non-ingestible.

5. The system of claim 1, wherein the substrate portion has a length from upstream end to downstream end of about 60 mm to about 110 mm.

6. The system of claim 1, wherein the substrate portion is tubular and has a cross-sectional diameter of about 5 mm to about 10 mm.

7. The system of claim 1 wherein the substrate portion has the form of a hollow tube.

8. The system of claim 1, wherein the substrate portion has the form of a rod comprising an air permeable material.

9. The system of claim 8, wherein the air permeable material comprises cellulose acetate tow or gathered non-woven polypropylene web.

10. The system of claim 8, wherein the rod is wrapped in a longitudinally-extending circumscribing paper wrap.

11. The system of claim 1, wherein the substrate portion has the form of a hollow tube having a plug of air permeable material disposed therein.

12. The system of claim 11, wherein the air permeable material comprises non-woven cellulose acetate fiber, cotton fibers, or open-cell foam.

13. The system of claim 1, further comprising an active ingredient incorporated within the substrate portion.

14. The system of claim 1, wherein the lozenge portion and substrate portion are in intimate contact.

15. The system of claim 1, wherein the lozenge portion and substrate portion are maintained in contact by friction fit between a surface of the lozenge portion and a surface of the substrate portion.

16. The system of claim 1, wherein the lozenge portion is positioned such that the downstream end of the lozenge portion is positioned downstream of the extreme downstream end of the substrate portion.

17. The system of claim 1, wherein the lozenge portion is positioned such that both ends of the lozenge portion are positioned upstream of the extreme downstream end of the substrate portion.

18. The system of claim 1, wherein the lozenge portion is positioned such that the downstream end of the lozenge portion is aligned with the extreme downstream end of the substrate portion.

19. The systems of claim 1, wherein the lozenge portion has a longitudinally extending length of about 4 mm to about 11 mm.

20. The system of claim 1, wherein the lozenge portion has a longitudinally extending length that is less than about 25 percent of the total length of the substrate.

21. The system of claim 1, wherein the lozenge portion has a volume of about 500 mm³ to about 2000 mm³.

22. The system of claim 1, wherein the lozenge portion has a generally cylindrical shape possessing a passageway therethrough.

23. A method of treating or delaying the progression of a condition, disease, or disorder responsive to activation of nicotinic acetylcholinergic receptors in a human subject, comprising administering a therapeutically effective amount of active ingredient in the form of the system of claim 1 to said human subject.

24. The method of claim 23, wherein said administering comprises administering the system to a human subject as a smoking cessation aid.
